(19) United States
(12) Patent Application Publication
(10) Pub. No.: US 2010/0036506 A1
(43) Pub. Date: Feb. 11, 2010

(54) Position Deviation Adjustment Apparatus For Prosthesis

(76) Inventor: Ya-Ping Wang, Taipei (TW)

Correspondence Address: LIN & ASSOCIATES INTELLECTUAL PROPERTY, INC., P.O. BOX 2339, SARATOGA, CA, 95070-0339, US

(21) Appl. No.: 12/188,212

(22) Filed: Aug. 8, 2008

(51) Int. Cl. A61F 2/62

(52) U.S. Cl. 623/38

(57) ABSTRACT

A position deviation adjustment apparatus for a prosthesis is provided. The position deviation adjustment apparatus includes a connecting member, a plate lid, an adjustment base, and a receptacle base. The connecting member is concentrically assembled with the plate lid. Then the assembled connecting member and plate lid together are assembled to the adjustment base. The adjustment base is then concentrically fixed to the receptacle base. When the connecting member and the plate lid are not yet fixed, they can be radially moved relative to the adjusting base, for aligning a center line of the connecting member and a center line of the receptacle base. Thereafter, the connecting member and the plate lid are fixed to the adjusting base.

### BACKGROUND OF THE INVENTION

1. Field of the Invention

The present invention relates generally to a position deviation adjustment apparatus for a prosthesis, and more particularly, to a position deviation adjustment apparatus having a movable and rotatable mechanism for adjusting relative positions of components of a prosthesis in accordance with a change of the barycenter of human body.

2. The Prior Arts

People with amputation due to severe trauma, vascular diseases, or congenital limb deficiency etc. need prostheses. FIG. 7 illustrates a major prosthetic assembly, which includes a receptacle barrel C, a connector A, and a pylon B having a receptacle base B1. The receptacle barrel C, the connector A, and the pylon B have their respective center lines C1, A1, and B2. Theoretically, the three central lines C1, A1, and B2 should be well-aligned with one another in the prosthetic assembly. However, in practical design, the three central lines C1, A1, and B2 have to be adjustable to accommodate different people's desires. The three central lines C1, A1, and B2 generally end up not so aligned with one another perfectly in practical use. Therefore, it is desired to design a prosthesis that can be adjusted in the three central lines according to patient's physical condition.

Referring to FIGS. 8 and 9, there is shown a conventional prosthesis structure having a position deviation adjustment apparatus. The conventional prosthesis structure includes a first structural element D, and a second structural element E. The first structural element is configured with a center line D1 and an opening D2. The first structural element has four sidewalls, each having a threaded hole D3 configured thereon. The second structural element E is integrally formed, including a round lid E1 jointed with a connecting member E2, a body E3, and a cone shaped member E4 configured on the body E3. The cone shaped member E4 of the second structural element E can be accommodated in the opening D2 of the first structural element D, and is adapted for forward, backward, leftward, rightward, and rotating movements inside the opening D2 of the first structural element D for aligning the center line E5 of the second structural element E with the center line C1 of the receptacle barrel C, and then driving four screws F to engage with the threaded holes D3 to check with the surface of the cone shaped member E4 so as to fix the second structural element E to the receptacle barrel C. When the receptacle base B1 is connected with the connecting member E2, the center line B2 of the pylon B can be correspondingly aligned with the center line C1 of the receptacle barrel C.

Although capable of solving the problem that the pylon B cannot be aligned with the receptacle barrel C, the foregoing position deviation adjustment apparatus unfortunately raises two critical safety problems. The first one safety problem is that because the cone shaped member E4 of the second structural element E is much smaller than the opening D2 of the first structural element D, when the second structural element E deviates relative to the opening D2, a screw F in one of the threaded holes D3 which is farther distant from the cone shaped member E4 will correspondingly extend longer than usual for checking surface of the cone shaped member E4. In this case, the screw F has much less area in contact with the threaded hole, and a slipped thread may occur therebetween, so that an end of the screw F may be unable to substantially check the surface of the cone shaped member E4 as desired, as shown in FIG. 9A, and further causes a looseness of the second structural element E, or even causes the problem that the center line B2 of the pylon B cannot be aligned with the center line C1 receptacle barrel C. The second one safety problem is that once the cone shaped member E4 is adjusted to a cornermost position as shown in FIG. 9B, all screws F are contacted with the cone shaped member E4 with the least contact areas, in which the cone shaped member E4 is least fixed by the screws F so that the cone shaped member E4 is likely to move or rotate and the user may fall down.

### SUMMARY OF THE INVENTION

A primary objective of the present invention is to provide a solution to the problem of the conventional position deviation adjustment apparatus for a prosthesis, that is the engagement between the second structural element and the receptacle barrel may become loose and disengaged when fixing these two elements.

For achieving the foregoing objective, the present invention provides a position deviation adjustment apparatus for a prosthesis. The position deviation adjustment apparatus includes a connecting member, a plate lid, an adjustment base, and a receptacle base. The connecting member is concentrically assembled with the plate lid. Then the assembled connecting member and plate lid together are assembled to the adjustment base. The adjustment base is then concentrically fixed to the receptacle base. When the connecting member and the plate lid are not yet fixed, they can be radially moved relative to the adjusting base, for aligning a center line of the connecting member and a center line of the receptacle base. Thereafter, the connecting member and the plate lid are fixed to the adjusting base.

Accordingly, the position deviation adjustment apparatus of the present invention includes a connecting member, a plate lid, an adjustment base, and a receptacle base. A round hollow pillar is provided over the connecting member. The round hollow pillar of the connecting member is inserted sequentially through a round through hole of the plate lid, and a longitudinal through hole of the adjusting base, and then locked by a fixing member. When the round hollow pillar of the connecting member is locked by the fixing member, the plate lid is firmly clamped by the adjustment base and the connecting member, and is prevented from radially moving or rotating. When the lock between the fixing member and the round hollow pillar of the connecting member is unlocked, the connecting member is allowed to radially move along a longitudinal direction of the longitudinal through hole, and the plate lid is allowed to move correspondingly. Meanwhile, the connecting member and the plate lid can be rotated relative to the adjustment base. The adjustment base further includes an adjustment base hollow pillar, for being assembled in an opening of the receptacle base, and locking the adjustment base to the receptacle base with screws.

The connecting member has a bottom end adapted for connecting with a first prosthesis structural element. The receptacle base has a top end adapted for connecting with a second prosthesis structural element. According to the present invention, the connecting member can be radially moved and rotated relative to the adjustment base, a relative position of the first prosthesis structural element and the second prosthesis structural element can be adjusted, so that the prosthesis user achieves a better wearing stability and comfort.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is an exploded side view of a position deviation adjustment apparatus for a prosthesis according to an embodiment of the present invention;

FIG. 2 is an exploded perspective view of the position deviation adjustment apparatus illustrating an assembling relationship between main elements thereof, according to an embodiment of the present invention;

FIG. 3 is an assembled side view of the position deviation adjustment apparatus according to an embodiment of the present invention;

FIG. 4 is an assembled cross-sectional view of the position deviation adjustment apparatus according to an embodiment of the present invention;

FIG. 5 is a cross-sectional view of the position deviation adjustment apparatus of FIG. 4 taken along line V-V;

FIG. 6 is a top view from a connecting member of the position deviation adjustment apparatus;

FIG. 7 is an exploded side view illustrating a set of conventional prosthesis components;

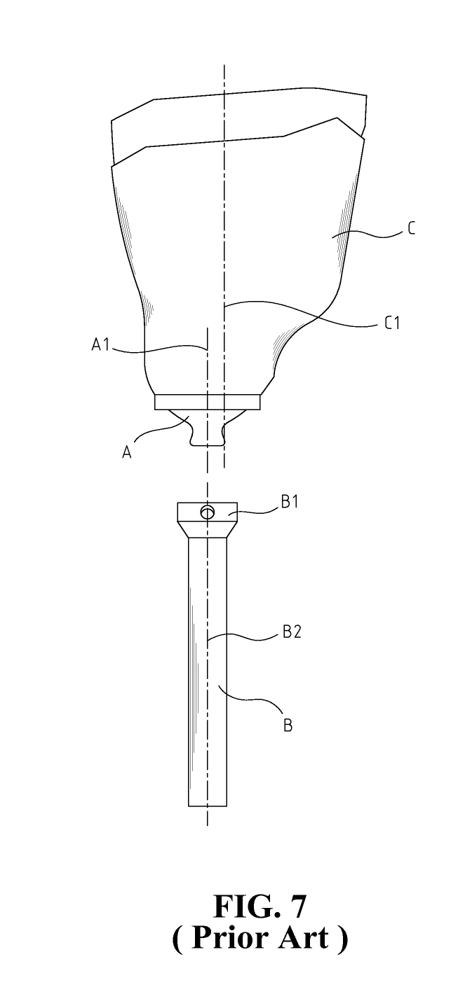

FIG. 8 is an exploded side view of a conventional position deviation adjustment apparatus;

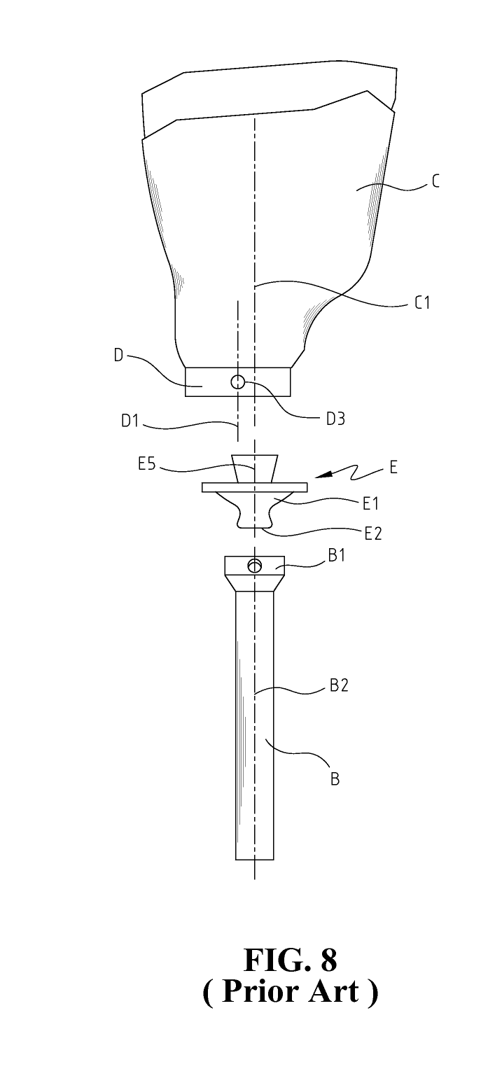

FIG. 9 is a cross-sectional view of the conventional position deviation adjustment apparatus of FIG. 8;

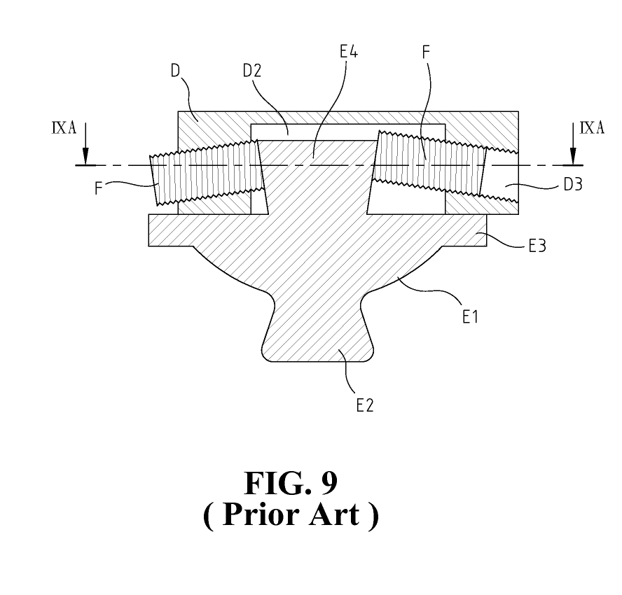

FIG. 9A is a cross-sectional view of FIG. 9 taken along line IXA-IXA; and

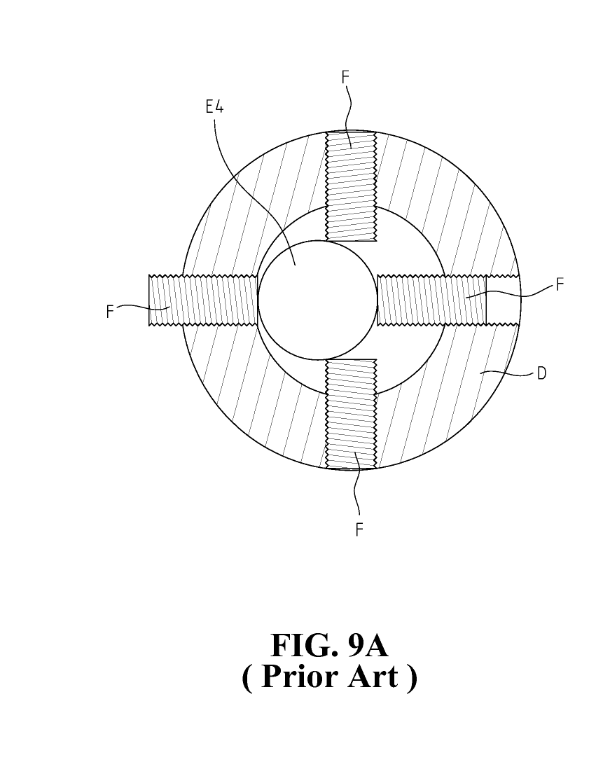

FIG. 9B is a schematic diagram illustrating the condition that the cone shaped member is adjusted to a cornermost position, and all screws are contacted with the cone shaped member with the least contact areas.

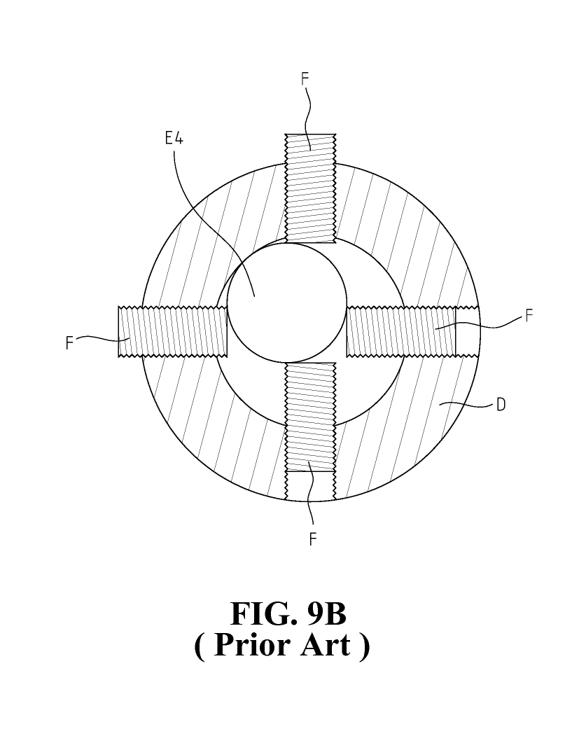

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

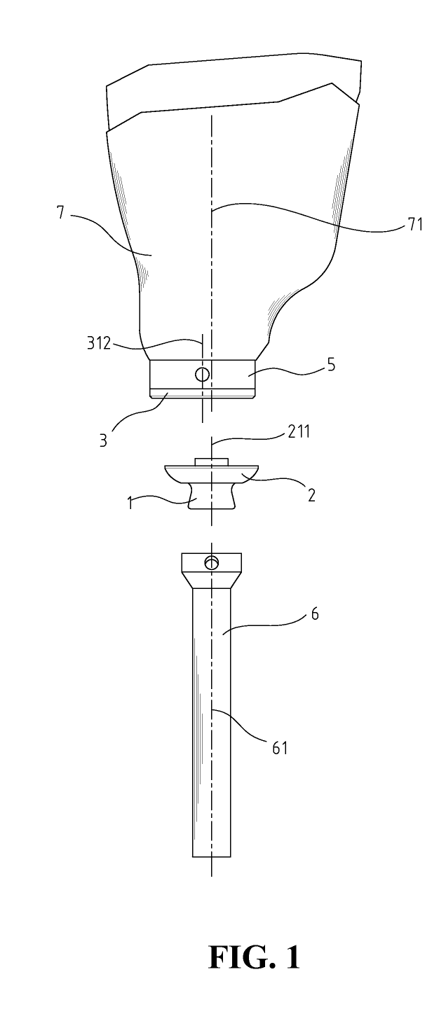

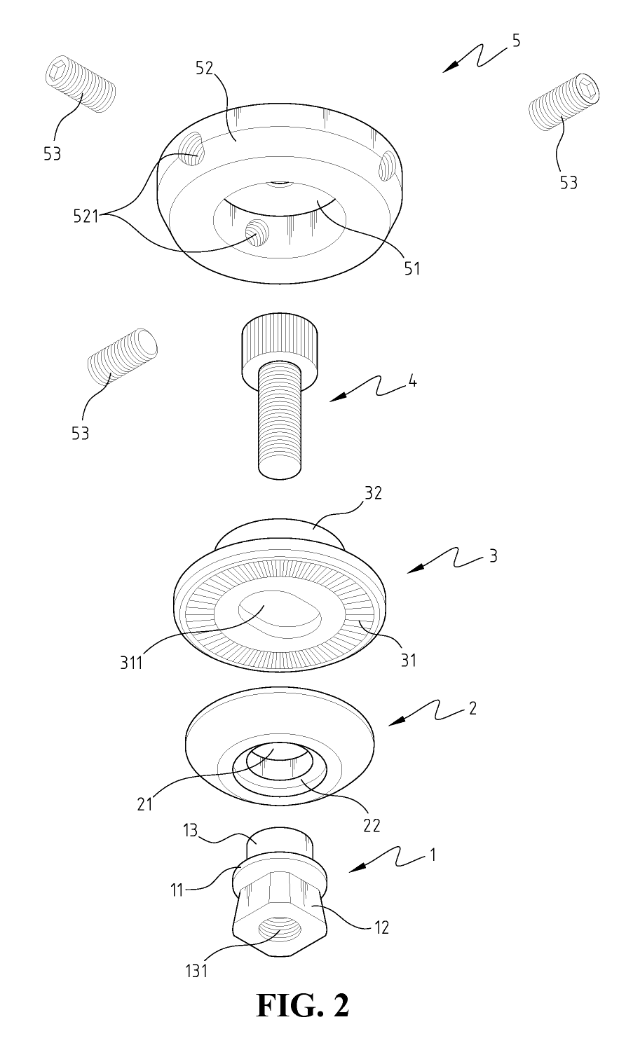

FIG. 1 is an exploded side view of a position deviation adjustment apparatus for a prosthesis according to an embodiment of the present invention. FIG. 2 is an exploded perspective view of the position deviation adjustment apparatus illustrating an assembling relationship between main elements thereof, according to an embodiment of the present invention. Referring to FIGS. 1 and 2, the position deviation adjustment apparatus for a prosthesis according to a preferred embodiment includes a connecting member 1, a plate lid 2, an adjustment base 3, a fixing member 4, and a receptacle base 5.

The connecting member 1 includes a middle portion 11 having a maximum diameter, a connection head 12 configured beneath the middle portion 11 for connecting with the a first prosthesis element 6, a hollow pillar 13 on the middle portion, and a threaded hole 131 extending from the hollow pillar 13 to the connection head 12 through a center part of the connecting member 1. Preferably, the hollow pillar 13 of the connecting member 1 is round shaped.

The plate lid 2 is configured with a through hole 21 having a shape corresponding to the shape of the hollow pillar 13 of the connecting member 1 at a center of the plate lid 2. The through hole 21 of the plate lid 2 is adapted to configure a loose fit with the hollow pillar 13 of the connecting member 1. The through hole 21 of the plate lid 2 is configured with a step edge 22 concentric with the through hole 21 at a peripheral edge of the through hole 21. According to an aspect of the embodiment, it is preferred to configure a plurality of saw teeth (not shown in the drawings) at an upper surface of the plate lid 2.

The adjustment base 3 includes a base side 31 having a diameter greater than the plate lid 2. The base side 31 is configured with a longitudinal through hole 311 extending along diametric direction of the base side 31 at a center of the base side 31. The longitudinal through hole 311 has a width slightly greater than an outer diameter of the hollow pillar 13 of the connecting member 1, so as to allow the hollow pillar 13 and the longitudinal through hole 311 configuring a loose fit. According to an aspect of the embodiment, the longitudinal through hole 311 can also be configured with a rectangular shape. The base side 31 is configured with a hollow pillar 32 of the adjustment base 3 at an end surface center. The hollow pillar 32 of the adjustment base 3 has a free end having a diameter greater than a diameter of a root portion of the hollow pillar 32, so that the hollow pillar 32 of the adjustment base 3 is configured with a cone shape. Preferably, the base side 31 is configured with a plurality of saw teeth, and the saw teeth of the base side 31 are adapted for meshing each other.

According to an embodiment of the present invention, the fixing member 4 is a screw which can be locked into the threaded hole 131 of the connecting member 1. The fixing member 4 can be locked into the threaded hole 131 of the connecting member 1 from an upside of the adjustment base 3, so as to assemble the connecting member 1, the plate lid 2 and the adjustment base 3 together.

The receptacle base 5 includes an opening 51 configured at a bottom side and having a certain depth. The depth and an inner diameter of the opening 51 are equivalent to a height and the outer diameter of the hollow pillar 32 of the adjustment base 3. The receptacle base 5 has a peripheral sidewall 52. The receptacle base 5 is further configured with a plurality of threaded holes 521 extending from an outside of the peripheral sidewall 52 to the opening 51. The threaded holes 521 are equidistantly distributed on the peripheral sidewall 52. Each of the threaded holes 521 is geared with a screw 53. An upper end of the receptacle base 5 is provided for connecting a second prosthesis element 7, as shown in FIG. 1.

The first prosthesis element 6 and the second prosthesis element 7, for example can be a calf prosthesis and thigh prosthesis, respectively, or a calf prosthesis and a sole prosthesis, respectively.

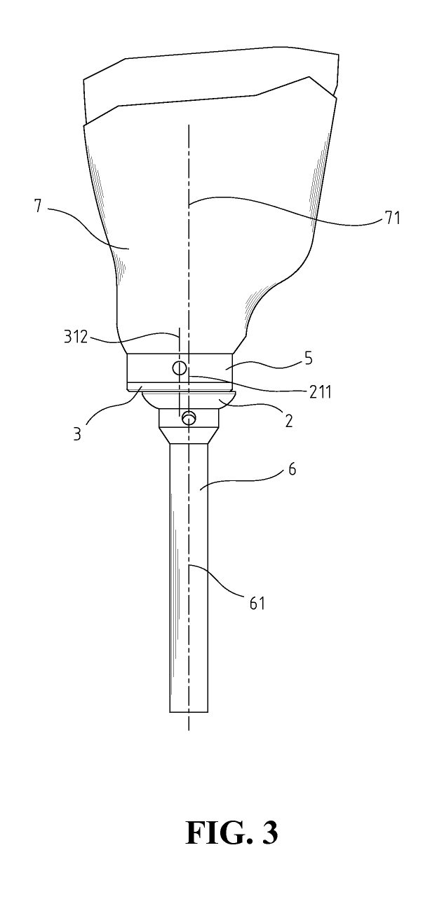

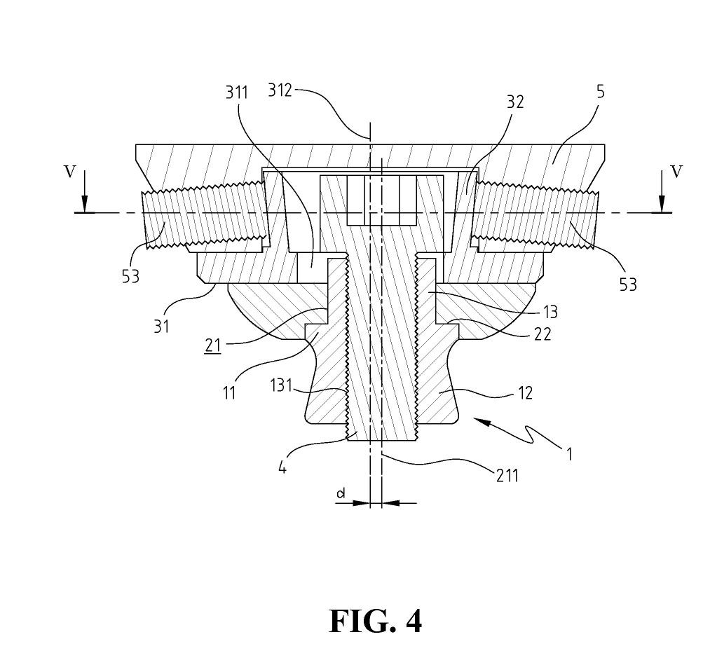

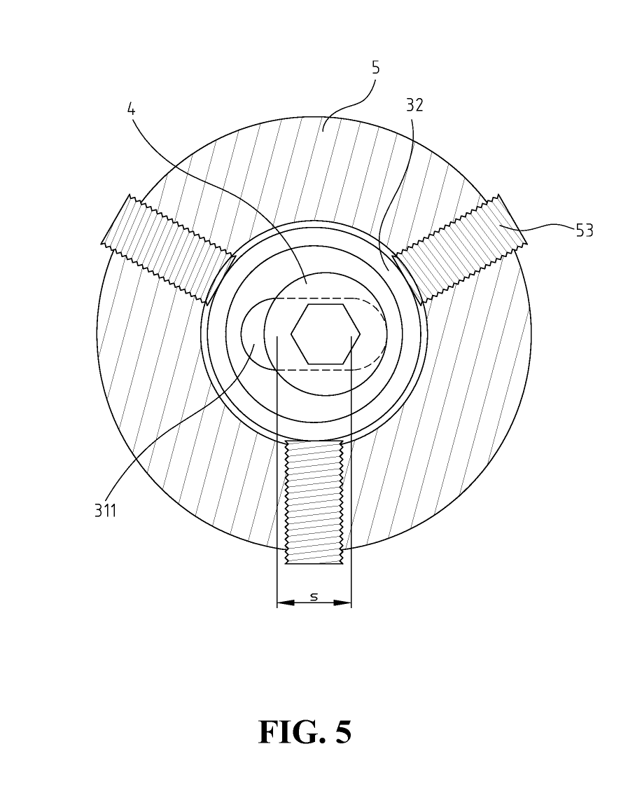

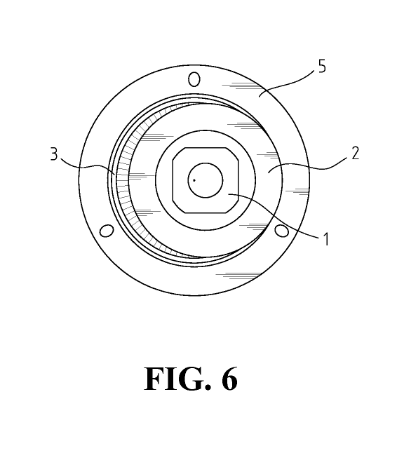

Referring to FIGS. 3 and 5, in assembling the foregoing elements, at first an upper end surface of the receptacle base 5 is fixed to the second prosthesis element 7, or other prosthesis elements connected with the second prosthesis element 7. Then, the connecting member 1, the plate lid 2 and the adjustment base 3 are assembled together. In assembling these three parts, the hollow pillar 13 of the connecting member 1 is inserted through the through hole 21 of the plate lid 2 until the middle portion 11 of the connecting member 1 reaches the step edge 22 of the plate lid. Then, the hollow pillar 13 of the connecting member 1 extending out from the upper end surface of the plate lid 2 is inserted through the longitudinal through hole 311 of the adjustment base 3. Then the fixing member 4 is locked into the threaded hole 131 of the connecting member 1. When the fixing member is not firmly fixed, the hollow pillar 13 of the connecting member 1 can be moved within a range S defined by the longitudinal through hole 311, by driving the middle portion of the connection member 1. In such a way, the plate lid 2 can be moved relative to a surface of the adjustment base 3, so that the center line 211 can achieve a positional deviation d from the center line 312 of the adjustment base 3, as shown in FIG. 4. Then, the fixing member 4 is firmly locked. The gearing between the saw teeth of the adjustment base 3 and the plate lid 2 prevents a relatively rotation therebetween, and maintains the positional deviation d between the plate lid 2 and the adjustment base 3. The adjustment base 3, together with the plate lid 2 and the connecting member 1 connected thereto, is assembled to the receptacle base 5, in which the upper end surface of the adjustment base 3 is overlapped with the bottom end surface of the receptacle base 5. Because the inner diameter of the opening 51 of the receptacle base 5 is greater than the outer diameter of the hollow pillar 32 of the adjustment base 3, the adjustment base 3 is allowed to rotate but not to translate relative to the receptacle base 5, as shown in FIGS. 5 and 6. As such, when the adjustment base 3 rotates, it drives the plate lid 2, the connecting member 1 to rotate together relative to the center line of the receptacle base 5. After rotating to adjust the position, screws 53 are geared in the threaded holes 521 one by one, so that the screws 53 check the outer surface of the hollow pillar 32 of the adjustment base 3. The outer diameter of the hollow pillar 32 of the adjustment base 3 is cone shaped, and when the hollow pillar 32 of the adjustment base 3 is received in the opening 51 of the receptacle base 5, the hollow pillar 32 of the adjustment base 3 is very close to the inner surface of the opening 51 of the receptacle base 5. Therefore, the hollow pillar 32 of the adjustment base 3 can be substantially uniformly disposed inside the opening 51, so as to allow the end surface of each screw 53 uniformly checking the outer surface of the hollow pillar 32 of the adjustment base 3. In such a way, the adjustment base 3 can be well fixed in the opening 51. And finally, the connection head 11 of the connecting member 1 is assembled to the first prosthesis element 6 to complete the assembling process.

Referring to FIGS. 3 through 6 again, in order to avoid muscle strain and muscle fatigue caused by long time wearing a prosthesis, the present invention configures a longitudinal through hole 311 on the adjustment base 3, which allows the hollow pillar 13 of the connecting member 1 moving along the longitudinal through hole 311. In such a way, the center line 61 of the first prosthesis element 6 connected to the connecting member 1 can be aligned with the center line 71 of the second prosthesis element 7. The adjustment base 3 can drive the connecting member 1 to rotate relative to the center line of the receptacle base 5, so as to maintain the center line 61 of the first prosthesis element 6 well aligned with the center line 71 of the second prosthesis element 7. Further, the present invention employs a plurality of threaded holes 521 equiangularly spaced at the receptacle base 5. Each of the threaded holes 521 is locked with a screw 53, for maintaining the screws 53 perpendicularly checking to the hollow pillar 32 of the adjustment base 3, so as to prevent slippery checking, unfirm fixing of the adjustment base 3. Specifically, according to an aspect of the embodiment, three threaded holes 521 are equiangularly configured at the receptacle base 5. Each of the threaded holes 521 is locked with a screw 53, so that the hollow pillar 32 of the adjustment base 3 is fixed by the three screws 53 in a manner of three-point fixing.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A position deviation adjustment apparatus for a prosthesis, comprising:
a connecting member, having:
a middle portion;
a connection head configured beneath the middle portion for connecting with a first prosthesis element;
a hollow pillar having a round shaped end surface configured on the middle portion; and
a threaded hole, extending from the hollow pillar of the connecting member to the connection head through a center part of the connecting member;
a plate lid, configured with a through hole having a shape corresponding to a shape of the hollow pillar of the connecting member at a center of the plate lid, wherein the through hole of the plate lid is configured with a step edge concentric with the through hole at a peripheral edge of the through hole;
an adjustment base, configured with
a longitudinal through hole which is configured axially through the adjustment base and radially extending, wherein the longitudinal through hole allows the hollow pillar to insert through; and
a hollow pillar of the adjustment base at a center of an upper surface of the adjustment base, wherein an inner diameter of the hollow pillar of the adjustment base is greater than a maximum length of the longitudinal through hole;
a fixing member, provided through the longitudinal through hole from the hollow pillar of the adjustment base to lock in the threaded hole of the hollow pillar of the connecting member; and
a receptacle base, configured with an opening at a bottom side for accommodating the hollow pillar of the adjustment base, the receptacle comprising a peripheral sidewall, and the receptacle base being configured with a plurality of threaded holes extending from an outside of the peripheral sidewall to the opening, wherein each of the threaded holes is geared with a screw, and when the screws locked in the openings, they check the hollow pillar of the adjustment base to firmly fix the hollow pillar of the adjustment base.

2. The position deviation adjustment apparatus according to claim 1, wherein the hollow pillar of the adjustment base is a cone shaped member having a free end with a diameter greater than a diameter of another end thereof.

3. The position deviation adjustment apparatus according to claim 1, wherein the plate lid and the adjustment base are contacted with contact surfaces respectively, and both of the contact surfaces of the plate lid and the adjustment base are configured with saw teeth thereon, and the saw teeth thereof are adapted for being meshed with one to another.
